(19) United States
(12) Patent Application Publication
(10) Pub. No.: US 2004/0220626 A1
(43) Pub. Date: Nov. 4, 2004

(54) Distributed subcutaneous defibrillation system

(76) Inventor: Darrell Orvin Wagner, Isanti, MN (US)

Correspondence Address: Crawford Maunu PLLC, Suite 390, 1270 Northland Drive, St. Paul, MN, 55120, US

(21) Appl. No.: 10/819,896

(22) Filed: Apr. 7, 2004

Related U.S. Application Data
(60) Provisional application No. 60/462,272, filed on Apr. 11, 2003.

(51) Int. Cl. A61N 1/39; A61N 1/362

(52) U.S. Cl. 607/4

(57) ABSTRACT

An implantable cardiac device includes first and second cans configured for subcutaneous, non-intrathoracic placement. Device circuitry is housed within and distributed between the first and second cans, including at least detection circuitry, energy delivery circuitry, and control circuitry. Communications circuitry may be included for communicating with a patient-external device. A lead may define a common potential and/or include a control line coupling the cans. Each of the first and second cans may include one or more sense and/or defibrillation electrodes. The cans may be coupled using only a wireless communications. Cardiac therapy may include bi-phasic or multi-phasic pulses concurrent, phased, and/or delayed between the two cans. Embodiments of methods in accordance with the present invention involve providing a first and second can of a cardiac therapy delivery device, each housing circuitry to deliver cardiac therapy concurrently, delayed and/or phased between the two cans.

### RELATED APPLICATIONS

This application claims the benefit of Provisional Patent Application Serial No. 60/462,272, filed on Apr. 11, 2003, to which priority is claimed pursuant to 35 U.S.C. §119(e) and which is hereby incorporated herein by reference.

### FIELD OF THE INVENTION

The present invention relates generally to subcutaneously implantable cardiac cardioverters/defibrillators and monitors and, more particularly, to subcutaneously implantable defibrillation systems with distributed circuitry.

### BACKGROUND OF THE INVENTION

When functioning properly, the human heart maintains its own intrinsic rhythm, and is capable of pumping adequate blood throughout the body's circulatory system. However, some people have irregular cardiac rhythms, referred to as cardiac arrhythmias. Such arrhythmias result in diminished blood circulation. One mode of treating cardiac arrhythmias uses drug therapy. Anti-arrhythmic drugs are often effective at restoring normal heart rhythms. However, drug therapy is not always effective for treating arrhythmias of certain patients. For such patients, an alternative mode of treatment is needed. One such alternative mode of treatment includes the use of a cardiac rhythm management system. Portions of such systems are often implanted in the patient and deliver therapy to the heart.

Implantable cardiac rhythm management systems have been used as an effective treatment for patients with serious arrhythmias. These systems typically include one or more leads and circuitry to sense signals from one or more interior and/or exterior surfaces of the heart. Such systems also include circuitry for generating electrical pulses that are applied to cardiac tissue at one or more interior and/or exterior surfaces of the heart. For example, leads extending into the patient's heart are connected to electrodes that contact the myocardium for sensing the heart's electrical signals and for delivering pulses to the heart in accordance with various therapies for treating arrhythmias.

Typical implantable cardioverter/defibrillators (ICDs) include one or more endocardial leads to which at least one defibrillation electrode is connected. Such ICDs are capable of delivering high-energy shocks to the heart, interrupting a ventricular tachyarrythmia or ventricular fibrillation, and allowing the heart to resume normal sinus rhythm. ICDs may also include pacing functionality.

Although ICDs are effective at preventing Sudden Cardiac Death (SCD), most people at risk of SCD are not provided with implantable defibrillators. The primary reasons for this unfortunate reality include the limited number of physicians qualified to perform transvenous lead/electrode implantation, a limited number of surgical facilities adequately equipped to accommodate such cardiac procedures, and a limited number of the at-risk patient population that may safely undergo the required endocardial or epicardial lead/electrode implant procedure. Subcutaneous ICDs are being developed to address these and other issues.

### SUMMARY OF THE INVENTION

The present invention relates generally to subcutaneously implantable cardiac cardioverters/defibrillators and monitors, and, more particularly, but not by way of limitation, to subcutaneously implantable defibrillation systems with distributed circuitry. An implantable cardiac device according to embodiments of the present invention includes a number of implantable cans, such as two or more implantable cans, wherein each of the cans is configured for subcutaneous, non-intrathoracic placement relative to a heart of a patient. Device circuitry is housed within and distributed between the various cans. The device circuitry includes at least detection circuitry, energy delivery circuitry, and control circuitry. The energy delivery circuitry is configured to deliver a cardiac therapy to the patient's heart.

According to one embodiment, an implantable cardiac device includes first and second cans configured for subcutaneous, non-intrathoracic placement relative to the heart of a patient. Device circuitry is housed within and distributed between the first and second cans, the device circuitry including at least detection circuitry, energy delivery circuitry, and control circuitry.

The device circuitry may include communications circuitry for effecting communications with a patient-external device. In a particular embodiment, the control circuitry and detection circuitry are housed in a first can, and the defibrillation and/or pacing energy delivery circuitry are housed in a second can.

A lead may be coupled to the first can and the second can, the lead including a conductor defining a common potential for the first and second cans. The lead may also include a control line and/or a power line that couples together the device circuitry respectively housed in the first and second cans. Each of the first and second cans may include one or more sense and/or defibrillation electrodes. The first can may be coupled to the second can using only a wireless communication coupling.

Power for the device circuitry may be supplied from one or more power sources. In one configuration, a single power source may be provided in one of the first and second cans. In another configuration, each of the first and second cans includes a power source. In a further configuration, a first power source is coupled to energy delivery circuitry (e.g., high voltage circuitry) and a second power source is coupled to at least detection and control circuitry (e.g., low voltage circuitry).

The cardiac therapy may include a bi-phasic or multi-phasic pulse, a first phase of the pulse provided by the first can and a second phase of the pulse provided by the second can. The control circuitry may provide a delay used to delay the energy delivery provided by the first can relative to the energy delivery provided by the second can.

In other embodiments of the present invention, a first and second can are configured for subcutaneous, non-intrathoracic placement relative to the heart of a patient. Device circuitry is housed within and distributed between the first and second cans. The device circuitry includes at least detection circuitry and control circuitry. A first energy storage device and first energy delivery circuitry are housed in the first can, coupled to the first energy storage device, and configured to deliver a cardiac therapy to the patient's heart.

The second energy delivery circuitry is housed within the second can and configured to deliver a cardiac therapy to the patient in coordination with the first energy delivery circuitry. The first and second energy delivery circuitry may be conductively coupled in series. An output of the first energy delivery circuitry is electrically coupled to at least a portion-of the first can, and an output of the second energy delivery circuitry is electrically coupled to at least a portion of the second can.

The first and second energy delivery circuitry may be conductively coupled in series and provide a combined output defibrillation voltage greater than about 800 Volts, for example, in one embodiment, and greater than about 1200 Volts and 2000 Volts in other embodiments. An output stage of the first and second energy delivery circuitry may include a current-limiting field-effect transistor, such as an insulated gate bipolar transistor. The first and second transistors may be configured to provide a combined output energy greater than about 40 Joules in one embodiment and an output energy greater than about 100 Joules in another embodiment.

The first can may be electrically coupled to the second can using a conductor, a potential of the conductor defining a common potential between the first can and the second can. In an alternate embodiment the cans may be coupled using only wireless communications.

The first energy delivery circuitry may further include a first output terminal, a first input terminal, and a first capacitor, with the first input terminal coupled to the first capacitor. A first switch may be coupled between the first input terminal and the first output terminal. The second energy delivery circuitry may further include a second output terminal, a second input terminal, and a second capacitor, with the second input terminal coupled to the second capacitor. A second switch may be coupled between the second input terminal and the second output terminal. The first output terminal may be electrically coupled to the second input terminal.

In another embodiment, the first energy delivery circuitry may include a first input terminal, a first output terminal, and a first capacitor, with a first switch and a second switch respectively coupled between the first input terminal and the first output terminal. The second energy delivery circuitry may include a second input terminal, a second output terminal, and a second capacitor, with a third switch and a fourth switch respectively coupled between the second input terminal and the second output terminal.

Embodiments of methods in accordance with the present invention involve providing a first and second can of a cardiac therapy delivery device configured for subcutaneous, non-intrathoracic placement relative to a heart of a patient. Each of the first and second cans house circuitry of the cardiac therapy delivery device. A cardiac therapy is delivered to the patient's heart using each of the first and second cans. The cardiac therapy may be delivered by cooperative use of the first and second cans, such as by delivering energy concurrently in time or in a time-phased relationship.

One of the first and second cans may define a first electrode for the cardiac therapy delivery device, and the other of the first and second cans may define a second electrode for the cardiac therapy delivery device. In another embodiment, a conductor may be coupled between the first and second cans and define an electrode of a second polarity for the cardiac therapy delivery device. The method may further involve detecting cardiac activity using one or both of the first and second cans, and delivering cardiac therapy from one or both cans. Power for the circuitry of the cardiac therapy delivery device may be supplied from one or both of the first and second cans.

The first and second cans may be positioned in relation to a heart so that a majority of ventricular tissue is included within a volume defined between the first and second cans, and may further be positioned such that the first can is in relation to a superior aspect of the heart and the second can is positioned in relation to an inferior aspect of the heart. One of the first and second cans may be positioned parallel to a ventricular free wall and extend a predetermined distance beyond an apex of the patient's heart.

Methods may further involve switchably coupling the first can to the second can for delivering the cardiac therapy, and delivering a shock to the patient's heart. The shock may have a potential greater than about 800 Volts, 1200 Volts, and 2000 Volts in various embodiments. The shock may have a total energy greater than about 40 Joules, 100 Joules, or 150 Joules in various embodiments.

The methods may also involve delivering a shock to the patient's heart, the shock including a multi-phasic pulse, wherein at least a first phase of the multi-phasic pulse is delivered by the first can and at least a second phase of the multi-phasic pulse is delivered by the second can.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

FIGS. 1A and 1B are views of a transthoracic cardiac sensing and/or stimulation device in accordance with the present invention as implanted in a patient;

FIG. 1C is a block diagram showing various components of a transthoracic cardiac sensing and/or stimulation device in accordance with an embodiment of the present invention;

FIG. 2 illustrates various components of a transthoracic cardiac sensing and/or stimulation device in accordance with an embodiment of the present invention;

FIG. 3A is a schematic drawing illustrating generally one can of a cardiac rhythm management device that employs multiple cans;

FIG. 3B is a schematic/block diagram illustrating generally an embodiment of portions of a defibrillation therapy circuit in accordance with the present invention; and

FIG. 4 illustrates various components of a distributed transthoracic cardiac sensing and/or stimulation device in accordance with an embodiment of the present invention.

While the invention is amenable to various modifications and alternative forms, specifics thereof have been shown by way of example in the drawings and will be described in detail below. It is to be understood, however, that the intention is not to limit the invention to the particular embodiments described. On the contrary, the invention is intended to cover all modifications, equivalents, and alternatives falling within the scope of the invention as defined by the appended claims.

### DETAILED DESCRIPTION OF VARIOUS EMBODIMENTS

In the following description of the illustrated embodiments, references-are made to the accompanying drawings, which form a part hereof, and in which is shown by way of illustration various embodiments in which the invention may be practiced. It is to be understood that other embodiments may be utilized, and structural and functional changes may be made without departing from the scope of the present invention.

A device employing an implantable cardiac device implemented in accordance with the present invention may incorporate one or more of the features, structures, methods, or combinations thereof described herein below. For example, a subcutaneous cardiac monitor or stimulator may be implemented to include components having one or more of the advantageous features and/or processes described below. It is intended that such a device or method need not include all of the features and functions described herein, but may be implemented to include selected features and functions that provide for unique structures and/or functionality.

In general terms, an implantable cardiac device implemented in accordance with the present invention may be used as a subcutaneous cardiac monitoring and/or stimulation device. One embodiment of the present invention is a distributed implantable transthoracic cardiac sensing and/or stimulation (DITCS) device that may be implanted under the skin in the chest region of a patient. The DITCS device may, for example, be implanted subcutaneously such that all or selected elements of the device are positioned on the patient's front, back, side, or other body locations suitable for sensing cardiac activity and delivering cardiac stimulation therapy. It is understood that elements of the DITCS device may be located at several different body locations, such as in the chest, abdominal, or subclavian region with electrode elements respectively positioned at different regions near, around, in, or on the heart.

In one embodiment of the present invention, the DITCS is configured with two or more housings (e.g., active cans) that, for example, may be configured for positioning outside of the rib cage at intercostal or subcostal locations, within the abdomen, or in the upper and lower chest regions. For example, a first can may be located at a subclavian location, such as above the third rib, and a second can may be located in the lower abdomen. Locations appropriate for implantation of subcutaneous cardiac devices and electrodes, and devices and methods for optimizing electrode orientations and locations useful in accordance with the present invention are further described in commonly assigned, co-pending U.S. patent application Ser. No. 10/465,520, filed Jun. 19, 2003 [Attorney Docket GUID.615PA] and U.S. Patent Application entitled “Electrode Placement Determination for Subcutaneous Cardiac Monitoring and Therapy,” filed Mar. 19, 2004 under Attorney Docket GUID.628PA, which are hereby incorporated herein by reference. Although embodiments of the present invention are described without leads attached to the can components, it is contemplated that leads having additional electrodes (e.g., subcutaneous array electrodes) and/or sensors may also be used with a distributed cardiac device without departing from the scope of the present invention.

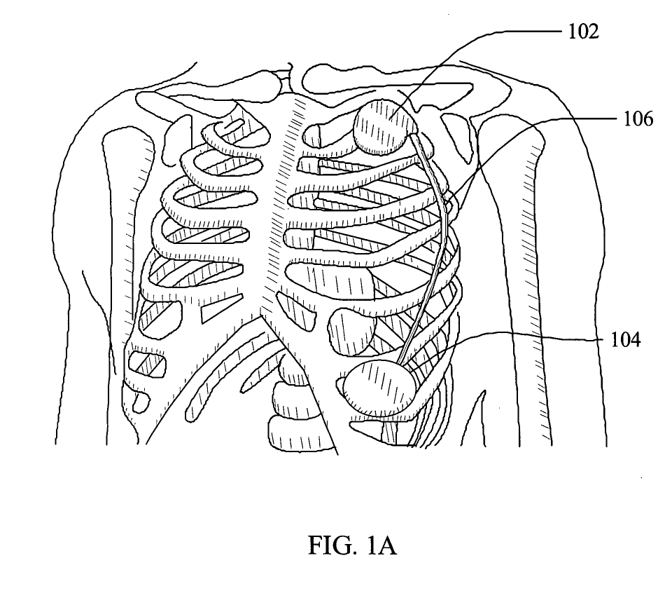

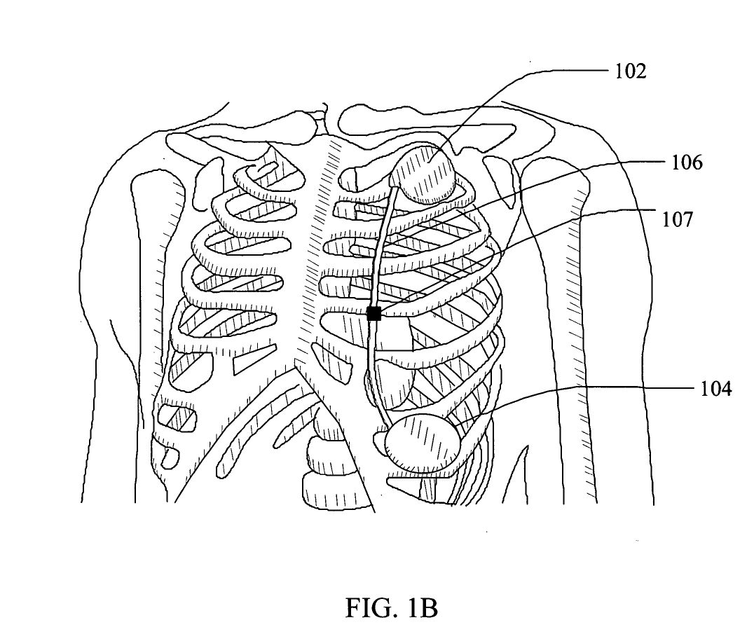

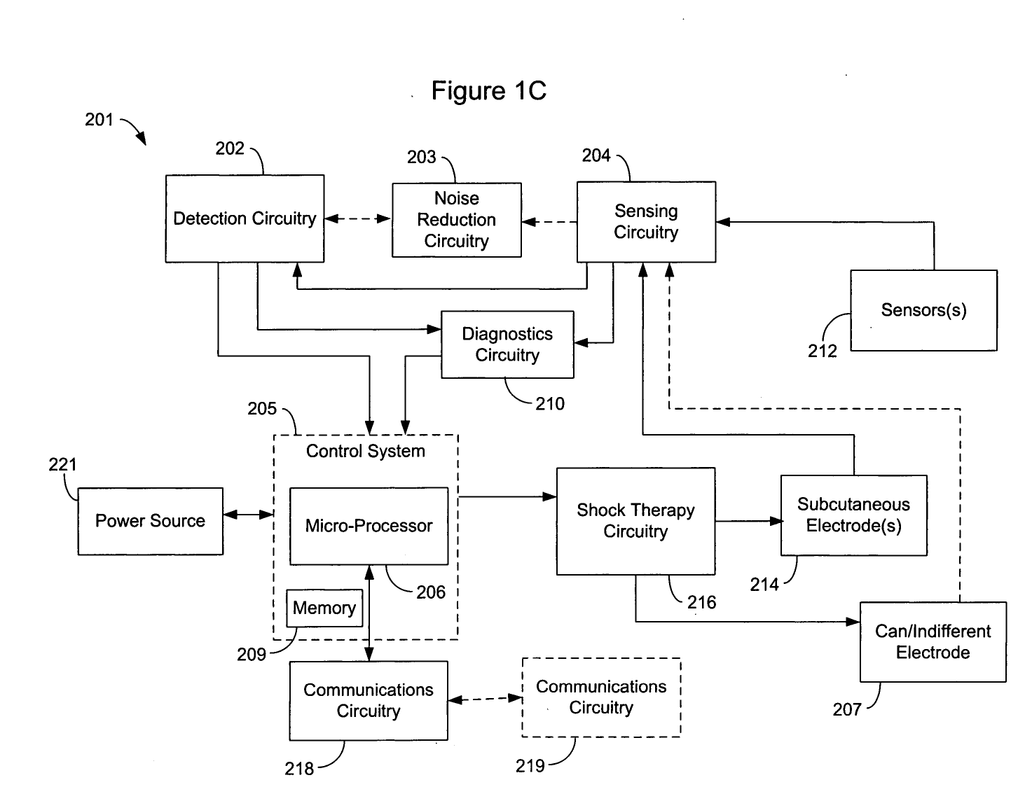

Referring now to FIGS. 1A and 1B of the drawings, there is shown a configuration of a DITCS device implanted in the chest region of a patient at different locations. In the particular configuration shown in FIGS. 1A and 1B, the DITCS device includes a first housing 102 within which various cardiac sensing, detection, processing, and energy delivery circuitry may be housed. The first housing 102 is typically configured to include one or more electrodes (e.g., can electrode and/or indifferent electrode).

In the configuration shown in FIGS. 1A and 1B, a second housing 104 may be positioned under the skin in the chest region and situated distal from the first housing 102. The first housing 102 and second housing 104 may be positioned about the heart at various locations and orientations, such as at various anterior and/or posterior locations relative to the heart. The second housing 104 is electrically coupled to circuitry within the first housing 102 via a lead, wire or cable 106. One or more conductors are provided by the lead, wire or cable 106 and electrically couple circuitry in the second housing 104 with circuitry in the first housing 102. One or both of control and power lines may be included within the lead, wire or cable 106. One or more sense, sense/pace or defibrillation electrodes may be situated on the lead, wire or cable 106. For example, a sense electrode 107, shown in FIG. 1B, may be provided on the lead, wire or cable 106. A DITCS system according to this approach is distinct from conventional approaches in that it may be configured to include a combination of two or more cans that are implanted subcutaneously.

In one configuration, the lead, wire or cable 106 is generally flexible. In another configuration, the lead, wire or cable 106 is constructed to be somewhat flexible, yet has an elastic, spring, or mechanical memory that retains a desired configuration after being shaped or manipulated by a clinician. For example, the lead, wire or cable 106 may incorporate a gooseneck or braid system that may be distorted under manual force to take on a desired shape. In this manner, the lead, wire or cable 106 may be shape-fit to accommodate the unique anatomical configuration of a given patient, and generally retains a customized shape after implantation. Shaping of the lead, wire or cable 106 according to this configuration may occur prior to, and during, DITCS device implantation.

In accordance with a further configuration, the lead, wire or cable 106 includes a support assembly, such as a rigid or semi-rigid elongated structure that positionally stabilizes the second housing 104 with respect to the first housing 102. In this configuration, the rigidity of the elongated structure is sufficient to maintain a desired spacing between the second housing 104 and the first housing 102, and a desired orientation of the second housing 104/first housing 102 relative to the patient's heart. The elongated structure may be formed from a structural plastic, composite or metallic material, and includes, or is covered by, a biocompatible material.

In one configuration, the support assembly, the first housing 102, and the second housing 104 define a unitary structure (i.e., a single housing/unit). The electronic components and electrode conductors/connectors are distributed within or on the unitary DITCS device housing/electrode support assembly. At least two electrodes are supported on the unitary structure near opposing ends of the housing/electrode support assembly, with appropriate electrical isolation provided. The unitary structure may have, for example, an arcuate or angled shape.

According to another configuration, the support assembly defines a physically separable unit relative to the first housing 102. The support assembly includes mechanical and electrical couplings that facilitate mating engagement with corresponding mechanical and electrical couplings of the first housing 102. For example, a header block arrangement may be configured to include both electrical and mechanical couplings that provide for mechanical and electrical connections between the support assembly and first housing 102. The header block arrangement may be provided on the first housing 102, the second housing 104, and/or the support assembly. Alternatively, a mechanical/electrical coupler may be used to establish mechanical and electrical connections between the support assembly and the first and second housings 102, 104. In such a configuration, a variety of different electrode support assemblies of varying shapes, sizes, and configurations can be made available for physically and electrically connecting to the DITCS device.

FIG. 1C is a block diagram depicting various components of a DITCS device 201 in accordance with embodiments of the present invention. The DITCS device 201 incorporates a processor-based control system 205 that includes a microprocessor 206 coupled to appropriate memory (volatile and/or non-volatile) 209, it being understood that any logic-based control architecture may be used. The control system 205 is coupled to circuitry and components to sense, detect, and analyze electrical signals produced by the heart and deliver electrical stimulation energy to the heart under predetermined conditions to treat cardiac arrhythmias.

Cardiac signals are sensed using the subcutaneous electrode(s) 214 and one or more can/indifferent electrode(s) 207 provided on one or both of the DITCS device 201 housings (for a two housing system). Cardiac signals may also be sensed using only the subcutaneous electrodes 214, such as in a non-active can configuration, or using only can electrodes 207 in, for example, a two-can system with each can having an electrode. As such, unipolar, bipolar, or combined unipolar/bipolar electrode configurations may be employed. The sensed cardiac signals are received by sensing circuitry 204, which includes sense amplification circuitry and may also include filtering circuitry and an analog-to-digital (A/D) converter. The sensed cardiac signals processed by the sensing circuitry 204 may be received by noise reduction circuitry 203, which may further reduce noise for signals used by the detection circuitry 202. Noise reduction circuitry 203 may also be incorporated after detection circuitry 202 in cases where high power or computationally intensive noise reduction algorithms are required.

In the illustrative configuration shown in FIG. 1C, the detection circuitry 202 is coupled to, or otherwise incorporates, noise reduction circuitry 203. The noise reduction circuitry 203 operates to improve the signal-to-noise ratio of sensed cardiac signals by removing noise content of the sensed cardiac signals introduced from various sources. Typical types of transthoracic cardiac signal noise includes electrical noise and noise produced from skeletal muscles, for example.

Detection circuitry 202 typically includes a signal processor that coordinates analysis of the sensed cardiac signals and/or other sensor inputs to detect cardiac arrhythmias, such as, in particular, tachyarrhythmia. Rate based and/or morphological discrimination algorithms may be implemented by the signal processor of the detection circuitry 202 to detect and verify the presence and severity of an arrhythmic episode. Examples of arrhythmia detection and discrimination circuitry, structures, and techniques, elements of which may be implemented by a DITCS device of a type contemplated herein, are disclosed in commonly owned U.S. Pat. Nos. 5,301,677 and 6,438,410, which are hereby incorporated herein by reference in their respective entireties.

The detection circuitry 202 communicates cardiac signal information to the control system 205. Memory circuitry 209 of the control system 205 contains parameters for operating in various sensing, defibrillation, and pacing modes, and stores data indicative of cardiac signals received by the detection circuitry 202. The memory circuitry 209 may also be configured to store historical ECG, blood information, and therapy data, which may be used for various purposes and transmitted to an external receiving device as needed or desired.

In certain configurations, the DITCS device 201 may include diagnostics circuitry 210. The diagnostics circuitry 210 typically receives input signals from the detection circuitry 202 and the sensing circuitry 204. The diagnostics circuitry 210 provides diagnostics data to the control system 205, it being understood that the control system 205 may incorporate all or part of the diagnostics circuitry 210 or its functionality. The control system 205 may store and use information provided by the diagnostics circuitry 210 for a variety of diagnostics purposes. This diagnostic information may be stored, for example, subsequent to a triggering event or at predetermined intervals, and may include system diagnostics, such as power source status, therapy delivery history, and/or patient diagnostics. The diagnostic information may take the form of electrical signals or other sensor data acquired immediately prior to therapy delivery.

According to a configuration that provides cardioversion and defibrillation therapies, the control system 205 processes cardiac signal data received from the detection circuitry 202 and initiates appropriate tachyarrhythmia therapies to terminate cardiac arrhythmic episodes and return the heart to normal sinus rhythm. The control system 205 is coupled to shock therapy circuitry 216. The shock therapy circuitry 216 is coupled to the subcutaneous electrode(s) 214 and/or the can electrodes 207 of the DITCS device 201 housings. Upon command, the shock therapy circuitry 216 delivers cardioversion and defibrillation stimulation energy to the heart in accordance with a selected cardioversion or defibrillation therapy. In a less sophisticated configuration, the shock therapy circuitry 216 is controlled to deliver defibrillation therapies, in contrast to a configuration that provides for delivery of both cardioversion and defibrillation therapies. Examples of ICD high energy delivery circuitry, structures and functionality, elements of which may be incorporated in a DITCS device of a type contemplated herein, are disclosed in commonly owned U.S. Pat. Nos. 5,372,606; 5,411,525; 5,468,254; 5,634,938; and 6,311,087, which are hereby incorporated herein by reference in their respective entireties.

The DITCS device 201 shown in FIG. 1C may be configured to receive signals from one or more physiologic and/or non-physiologic sensors. Depending on the type of sensor employed, signals generated by the sensors may be communicated to transducer circuitry coupled directly to the detection circuitry or indirectly via the sensing circuitry. It is noted that certain sensors may transmit sense data to the control system 205 without processing by the detection circuitry 202.

Communications circuitry 218, 219 is coupled to the microprocessor 206 of the control system 205. The communications circuitry 218, 219 allows the DITCS device 201 to effect communications between multiple housings having distributed componentry, and with one or more receiving devices or systems situated external to the DITCS device 201. By way of example, the DITCS device 201 may communicate with a patient-worn, portable or bedside communication system via the communications circuitry 218, 219. In one configuration, one or more physiologic or non-physiologic sensors (subcutaneous, cutaneous, or external of patient) may be equipped with a short-range wireless communication interface, such as an interface conforming to a known communications standard, such as Bluetooth or IEEE 802 standards. Data acquired by such sensors may be communicated to the DITCS device 201 via the communications circuitry 218, 219. It is noted that physiologic or non-physiologic sensors equipped with wireless transmitters or transceivers may communicate with a receiving system external of the patient.

In another example, a short-range wireless communication interface, such as an interface conforming to a known communications standard, such as Bluetooth or IEEE 802 standards, may be used to coordinate therapy between multiple housings of the DITCS device 201, where the housings may be coupled only using wireless communications. For example, communications circuitry 218 may reside in a first housing and communications circuitry 219 may reside in a second housing. In cases where only wireless communications are used, each housing of the DITCS device 201 may also have its own power supply.

In one implementation, each housing includes componentry needed to operate autonomously as a subcutaneous transthoracic defibrillator, wherein cooperative operation among the autonomous ITCS devices is facilitated via wireless communications. In another similar implementation, each housing includes componentry needed to autonomously operate limited or specific functions of a subcutaneous transthoracic defibrillator, such as one or two housings performing energy delivery functions, while another one or two housings perform sensing and detection functions.

The communications circuitry 218, 219 may also allow the DITCS device 201 to communicate with an external programmer. In one configuration, the communications circuitry 218, 219 and the programmer unit (not shown) use a wire loop antenna and a radio frequency telemetric link, as is known in the art, to receive and transmit signals and data between the programmer unit and communications circuitry 218, 219. In this manner, programming commands and data are transferred between the DITCS device 201 and the programmer unit during and after implant. Using a programmer, a physician is able to set or modify various parameters used by the DITCS device 201. For example, a physician may set or modify parameters affecting sensing, detection, and defibrillation functions of the DITCS device 201, including cardioversion/defibrillation therapy modes.

Typically, the DITCS device 201 is encased and hermetically sealed in two or more housings suitable for implanting in a human body. Power to the DITCS device 201 is supplied by one or more electrochemical power source(s) 221 housed within the DITCS device 201. In one configuration, the power source(s) 221 include a rechargeable battery. According to this configuration, charging circuitry is coupled to the power source(s) 221 to facilitate repeated non-invasive charging of the power source(s) 221. The communications circuitry 218, 219, or separate receiver circuitry, is configured to receive RF energy transmitted by an external RF energy transmitter. The DITCS device 201 may, in addition to a rechargeable power source, include a non-rechargeable battery. It is understood that a rechargeable power source need not be used, in which case a long-life non-rechargeable battery is employed.

It is understood that the components illustrated in FIG. 1C may be distributed among several enclosures of a DITCS device, and that some or all of the components may be situated in such as way as to be included in only one of the enclosures. Alternatively, some or all of the components illustrated in FIG. 1C may be situated in each of the enclosures, thereby effectively populating these components in each of the enclosures.

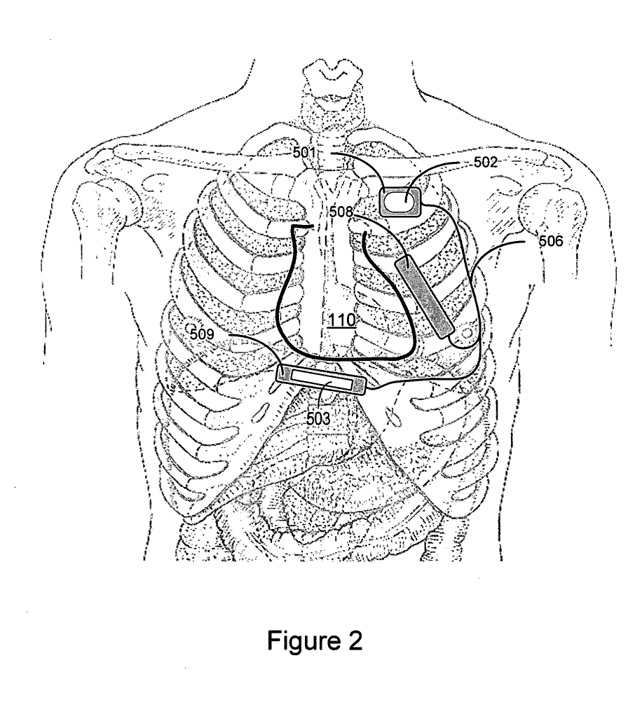

Referring to FIG. 2, a can electrode 502 is positioned on a housing 501 that encloses at least a portion of the DITCS device electronics. A can electrode 503 is positioned on a housing 509 that also encloses at least a portion of the DITCS device electronics. In one embodiment, the can electrodes 502, 503 include the entirety of the external surfaces of housings 501, 509. In other embodiments, various portions of the housings 501, 509 may be electrically isolated from the can electrodes 502, 503 or from tissue. For example, the active area of the can electrodes 502, 503 may include all or a portion of either the anterior or posterior surfaces of the housings 501, 509 to direct current flow in a manner advantageous for cardiac sensing and/or stimulation.

The housings 501, 509 may resemble that of a conventional implantable ICD, and may be approximately 20-100 cc in volume, with a thickness of 0.4 to 2 cm and with a surface area on each face of approximately 20 to 100 cm². For example, each of the housings 501, 509 may be equivalent in size to a conventional single-can device or smaller. As previously discussed, portions of the housings 501, 509 may be electrically isolated from tissue to optimally direct current flow. For example, portions of the housings 501, 509 may be covered with a non-conductive, or otherwise electrically resistive, material to direct current flow. Suitable non-conductive material coatings include coatings formed from silicone rubber, polyurethane, polyvinylidene fluoride, or parylene, for example.

In addition, or alternatively, all or portions of the housings 501, 509 may be treated to change the electrical conductivity characteristics thereof for purposes of optimally directing current flow. Various known techniques can be employed to modify the surface conductivity characteristics of the housings 501, 509, such as by increasing or decreasing surface conductivity, to optimize current flow. Such techniques may include mechanically or chemically altering the surface of the housings 501, 509 to achieve desired electrical conductivity characteristics.

In the configuration shown in FIG. 2, one or both of the DITCS device housings 501, 509 containing the electronics may or may not be used as an electrode. In the DITCS device illustrated in FIG. 2, an (optional) lead system may include an electrode subsystem 508 coupled to the housings 501, 509, and may be implanted subcutaneously in the chest region of the body, such as in the anterior thorax.

If both the housings 501, 509 employ an active electrode portion, the housings 501, 509 are placed in opposition with respect to the ventricles of a heart 110, with the majority of the ventricular tissue of the heart 110 included within a volume defined between the housings 501, 509. As illustrated in FIG. 2, the first housing 501 is positioned superior to the heart 110. The second housing 509 is located inferior to the heart 110 and positioned in relation to an inferior aspect of the heart 110, e.g., parallel to the right ventricular free wall. A cable or wiring 506 conductively couples the electrode subsystem 508 to the housing 501, and may (optionally) couple the electrode subsystem 508 to the housing 509 or both of the housings 501, 509.

In this configuration, the first and the second housings 501, 509 and the (optional) electrode subsystem 508 may include any combination of electrodes used for sensing and/or electrical stimulation. In various configurations, the electrode subsystem 508 may include a single electrode or a combination of electrodes. The electrode or electrodes in the electrode subsystem 508 may include any combination of one or more coil electrodes, tip electrodes, ring electrodes, multi-element coils, spiral coils, spiral coils mounted on non-conductive backing, and screen patch electrodes, for example.

An important design requirement for a cardiac rhythm management system is ensuring delivery of a defibrillation shock having sufficient energy to terminate a tachyarrhythmia or cardiac fibrillation. By way of example, a transformer-coupled dc-to-dc voltage converter may be designed to transform a battery voltage (e.g., battery voltages approximately between 1.5 Volts and 6.5 Volts) up to a high defibrillation voltage (e.g., defibrillation voltages possibly up to 1000 Volts). The energy associated with this high defibrillation voltage is typically stored in a storage capacitor. A defibrillation energy delivery circuit delivers the defibrillation energy from the storage capacitor to defibrillation lead wires and defibrillation electrodes associated with the heart. Upon receiving this defibrillation energy via the defibrillation electrodes, the heart resumes normal rhythms if the defibrillation therapy is successful.

Subcutaneous defibrillation energy requirements may be substantially greater than those associated with conventional ICDs that use intracardiac leads and electrodes. Energy requirements may be as high as, or possibly higher than, 100 Joules in some subcutaneous applications. Also, a greater safety margin may be necessary to assure that ventricular arrhythmias can be safely defibrillated in patients having varying defibrillation thresholds. To achieve such energies, conventional ICDs using voltages at about 700 Volts and having capacitors rated at 150 uF would typically not meet the requirements associated with subcutaneous transthoracic defibrillation.

Metal Oxide Semiconductor Field Effect Transistors (MOSFETs) may be used for switching on and off the defibrillation shock waveform, but are limited in terms of maximum voltage levels. Certain technologies such as Insulated Gate Bipolar Transistor (IGBT) MOSFETs, however, are rated at higher voltages, such as at 1200 Volts. Even if IGBT MOSFETs were incorporated into a subcutaneous ICD device, the total energy capacity would be limited to about 108 Joules when using 1200 Volts and 150 uF. In addition, some safety margin would need to be given the IGBT MOSFETs that would necessitate them working at less the 1200 Volts, such as 1150 Volts giving only about 99 Joules.

To obtain 150 Joules for an example, the voltage would need to be 1414 Volts at 150 uF. Even if MOSFETs could be acquired that could handle 1500 Volts, the other components associated with the subcutaneous device would need to be able to withstand the high voltage, such components including charging circuitry, capacitors, and dielectric spacing for individual components. Given these considerations, the ICD enclosure would, by necessity, have to be made sufficiently large in order to accommodate these requirements, where space is at a premium for implantable devices.

A DITCS device implemented in accordance with the present invention advantageously enables the size or bulk volume of a subcutaneous ICD to be effectively spread out over two of more enclosures. For example, the size of each enclosure may be reduced by a factor corresponding to the number of enclosures that make up the DITCS device. In a two-can DITCS device implementation, for example, the size or bulk volume of each enclosure may be reduced by at least one-half that of a one-enclosure system if spread out equally. A one-third size reduction (or bulk volume reduction) may be realized in a three-can DITCS device implementation. Componentry of a DITCS device may be distributed equally or unequally among two or more enclosures. In addition, the high voltage electronics may be distributed between enclosures so that maximum voltage levels are lowered in each enclosure, thereby enabling the use of smaller components and closer component spacing—all contributing to smaller individual enclosures.

A DITCS device of the present invention enables a subcutaneous defibrillation system to be less bulky, and instead, distributed over the area of defibrillation, giving a more acceptable form and patient comfort. For design purposes, maximum voltage levels may be reduced in each enclosure if the high voltage electronics are divided between enclosures or the hardware may be distributed as to function, thereby enhancing flexibility.

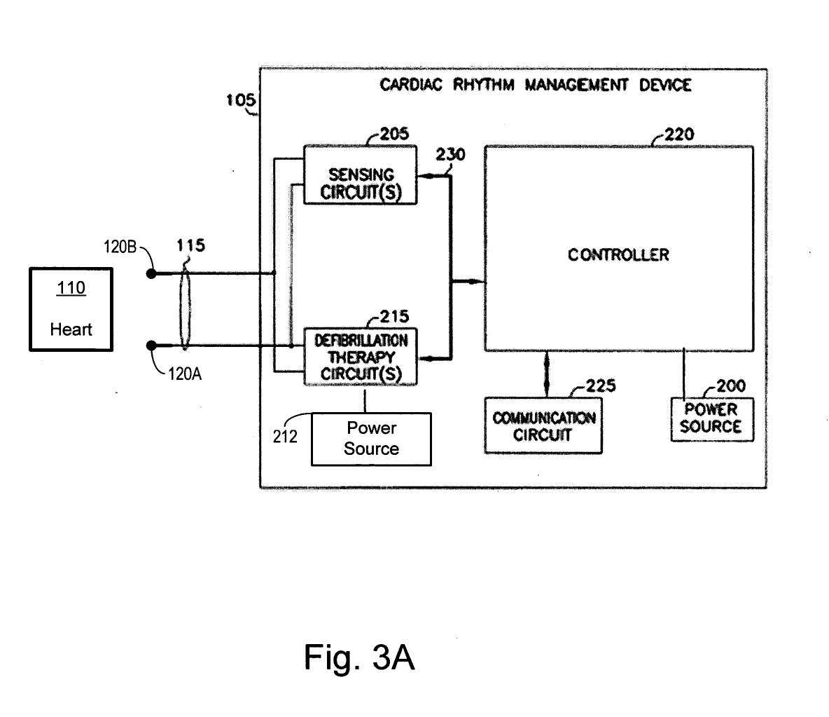

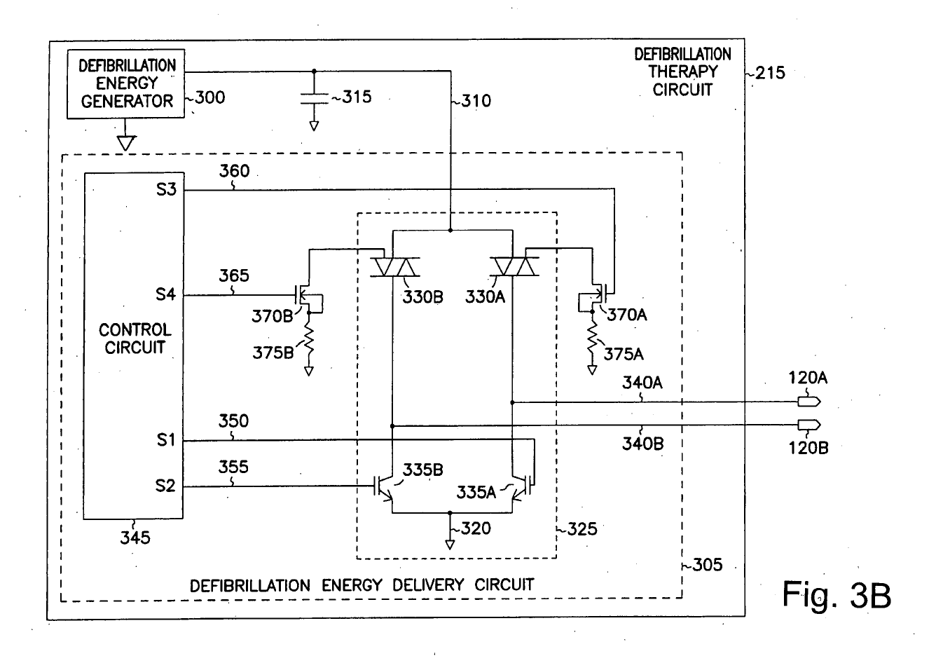

FIG. 3A is a schematic diagram illustrating an embodiment of portions of an implantable DITCS device 105, which is conductively coupled to the heart 110. The implantable DITCS device 105 includes a power source 200 and a power source 212, heart signal sensing circuit(s) 205, defibrillation therapy circuit(s) 215, a controller 220, and a communication circuit 225 for communicating with a programmer via a telemetry device.

The heart signal sensing circuits 205 are coupled by one or more cables or wiring 115 to two or more electrodes positioned about the heart 110 for receiving, sensing, and/or detecting electrical heart signals. Atrial and/or ventricular heart signals may be sensed by the sensing circuits 205. Sensing circuits 205 facilitate sensing and detection of normal rhythms and abnormal rhythms, including tachyarrhythmias, such as fibrillation, and other activity. Sensing circuits 205 provides one or more signals to controller 220, via a node/bus 230, based on the received heart signals.

In one embodiment, defibrillation therapy circuit(s) 215 provide cardioversion/defibrillation therapy, as appropriate, to cardiac electrodes 120A, 120B. Controller 220 controls the delivery of defibrillation therapy by defibrillation therapy circuit(s) 215 based on heart activity signals received by sensing circuit(s) 205. Controller 220 may further control the delivery of pacing therapy, such as by use of a pacing therapy circuit (not shown). Controller 220 includes various modules, which are implemented either in hardware or as one or more sequences of steps carried out on a microprocessor or other controller. Such modules may be conceptualized separately, but it is understood that the various modules of controller 220 need not be separately embodied, but may be combined and/or otherwise implemented, such as in software/firmware.

In general terms, sensing circuit(s) 205 sense electrical signals from the heart 110. Sensing circuit(s) 205 and/or controller 220 process these sensed signals. Based on the sensed signals, controller 220 issues control signals to therapy circuit(s) 215, if necessary, for the delivery of electrical energy (e.g., defibrillation pulses) to the appropriate electrodes using cables or wiring 115. Controller 220 may include a microprocessor or other controller for execution of software and/or firmware instructions. The software of controller 220 may be modified (e.g., by remote external programmer 150) to provide different parameters, modes, and/or functions for the implantable DITCS device 105 or to adapt or improve performance of the implantable DITCS device 105.

As described previously, the circuitry of the implantable DITCS device 105 is distributed between two or more housings or cans. For example, a first housing may contain the power source 212 and all or a portion of the defibrillation therapy circuit(s) 215, while a second housing contains the power source 200, the controller 220 and the remaining circuitry. The cables or wiring 115 may then provide a common reference potential between the two or more housings. If the defibrillation therapy circuit(s) 215 of the first housing and second housing are connected in series, using the cables or wiring 115, the effective voltage (energy, current) of the implantable DITCS device 105 may be double that of a single defibrillator of the prior art.

FIG. 3B is a schematic/block diagram illustrating one housing of an implantable DITCS device in accordance with the present invention. In FIG. 3B, defibrillation therapy circuit 215 includes a defibrillation energy generator circuit 300 and a defibrillation energy delivery circuit 305. In one example, defibrillation energy generator 300 includes a transformer-coupled dc-to-dc voltage converter that transforms a voltage provided by power source 200 shown in FIG. 3A (e.g., source voltages of approximately between 1.5 Volts and 6.5 Volts) up to a high defibrillation voltage (e.g., defibrillation voltages up to approximately 1000 Volts) at node 310. The energy associated with this high defibrillation voltage is typically stored on a defibrillation energy storage capacitor 315 until delivered to the heart. While the defibrillation voltages of up to approximately 1000 Volts generally describe current applications for a single can system, defibrillation voltages may often fall within the range of approximately 600 to 800 Volts, such as approximately 780 Volts or approximately 645 Volts.

Defibrillation energy delivery circuit 305 includes a first input terminal that receives a first high voltage (e.g., the voltage associated with the defibrillation energy at node 310) and a second input terminal that receives a second high voltage (e.g., the high voltage common at node 320). Energy delivery circuit 305 includes an H-bridge 325, which includes four switches (i.e., first and second pull-up switches 330A-B and first and second pull-down switches 335A-B) for coupling the defibrillation energy at node 310 and the high voltage common at node 320 to first and second output terminals at nodes 340A-B, respectively.

Pull-up switches 330A-B couple the defibrillation energy at node 310 to respective first and second output terminals at nodes 340A-B, respectively. Pull-down switches 335A-B couple the high voltage common at node 320 to respective first and second output terminals at nodes 340A-B, respectively. In one embodiment, for delivering a monophasic defibrillation energy pulse, control circuit 345 (which may alternatively be conceptualized as part of controller 220 shown in FIG. 3A) turns on pull-down switch 335A, then turns on pull-up switch 330B. To discontinue energy delivery, control circuit 345 turns off pull-down switch 335A, thus turning off pull-up switch 330B. To further deliver a biphasic defibrillation energy pulse, control circuit 345 then turns on pull-down switch 335B, then turns on pull-up switch 330A. To discontinue energy delivery, control circuit 345 then turns off pull-down switch 335B, thus turning off pull-up switch 330A.

Multiphasic energy delivery may be generated by repeating the cycle of delivering and discontinuing an energy pulse in similar fashion for three or more cycles. It is thus inherent that the two cycles of biphasic energy delivery are a subset of the three or more cycles of multiphasic energy delivery. In one embodiment, the user may select between monophasic, biphasic or multiphasic energy delivery using a programmer to program an implantable DITCS device to operate accordingly.

A programmer may also be used to program the implantable DITCS device to operate with delay or phased output between multiple cans and/or electrodes. For example, a phased output between two or more cans of a multi-can DITCS system may provide increased efficacy, by providing a superposition of energy in the patient's heart, or by providing energy delivery in a phased relationship within different portions of the patient's heart.

Pull-down switches 335A-B may include, for example, insulated gate bipolar transistors (IGBTs) or other switching devices that couple the respective first and second defibrillation outputs 120A, 120B to high voltage common node 320. In one embodiment, first pull-down switch 335A includes a collector coupled to first defibrillation electrode 120A, a gate coupled to receive control signal S1 at node 350 from control circuit 345, and an emitter coupled to high voltage common node 320. Second pull-down switch 335B includes a collector coupled to second defibrillation electrode 120B, a gate coupled to receive control signal S2 at node 355 from control circuit 345, and an emitter coupled to high voltage common node 320.

First and second pull-up switches 330A-B may include, for example, triacs, thyristors, semiconductor-controlled rectifiers (SCRs), semiconductor-controlled switches (SCSs), four-layer diodes or other switching devices that couple the high voltage associated with the defibrillation energy at node 310 to first and second defibrillation outputs 120A, 120B, respectively. In one embodiment, control signals S3 and S4 are received at nodes 360 and 365, respectively, from control circuit 345 to initiate operation of first and second pull-up switches 330A-B, respectively. In one embodiment, control signals S3 and S4 activate triggering devices 370A-B, which provide triggering signals to the gates of switches 330A-B, respectively

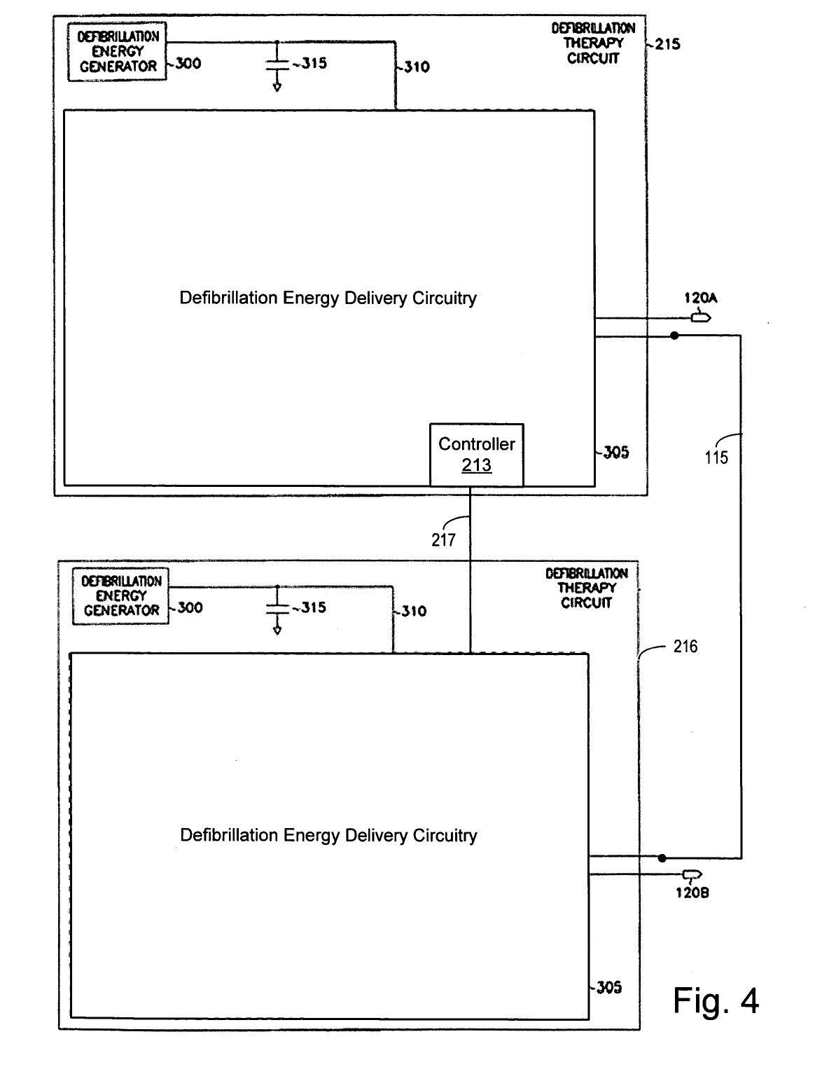

Referring now to FIG. 4, an implantable DITCS device is illustrated having a defibrillation therapy circuit 215 connected in series to a defibrillation therapy circuit 216 in accordance with embodiments of the present invention. Cables or wiring 115 connect the high voltage common potential output of defibrillation therapy circuit 215 (consistent with the second defibrillation output 120B of FIG. 3A and 3B) to the high potential output of defibrillation therapy circuit 216 (consistent with defibrillation output 120A of FIGS. 3A and 3B).

A single controller 213 may be used, or a controller in each of the defibrillation therapy circuit 216 and the defibrillation therapy circuit 215 may coordinate therapy using a communications link 217. Using the configuration of the defibrillation therapy circuit 215 and the defibrillation therapy circuit 216 illustrated in FIG. 4, the potential developed between the defibrillation output 120A and the defibrillation output 120B is potentially twice that capable of being produced by a single can, such as illustrated in FIG. 3B. The schematics and block diagrams illustrated in FIGS. 3A, 3B, and 4 may incorporate circuitry and functionality described in commonly owned U.S. Pat. No. 6,311,087, which has previously been incorporated by reference herein.

The implantable DITCS device illustrated in FIG. 4 is capable of defibrillation voltages of up to approximately 2000 Volts. Defibrillation voltages of the implantable DITCS device in accordance with the present invention may more typically fall within the range of approximately 800 to 1600 Volts. Similarly to the voltage increase available from implantable DITCS devices in accordance with the present invention, the energy available may also be doubled for a two-can system. For example, a single system may be capable of delivering a maximum of only 100 Joules of energy in a shock, whereas a system such as is illustrated in FIG. 4 may be capable of delivering up to 200 Joules of energy in a shock. It is also contemplated that other configurations, such as two-can parallel systems, may provide twice the energy at similar voltages in comparison to conventional systems.

Various modifications and additions can be made to the preferred embodiments discussed hereinabove without departing from the scope of the present invention. Accordingly, the scope of the present invention should not be limited by the particular embodiments described above, but should be defined only by the claims set forth below and equivalents thereof.

What is claimed is:

1. An implantable cardiac device, comprising;
a plurality of implantable cans, each of the cans configured for subcutaneous, non-intrathoracic placement relative to a heart of a patient; and
device circuitry housed within and distributed between the plurality of cans, the device circuitry comprising at least detection circuitry, energy delivery circuitry, and control circuitry, the energy delivery circuitry configured to deliver a cardiac therapy to the patient's heart.

2. The device of claim 1, wherein the device circuitry comprises communications circuitry for effecting communications with a patient-external device.

3. The device of claim 1, wherein the energy delivery circuitry comprises defibrillation energy delivery circuitry.

4. The device of claim 1, wherein the energy delivery circuitry comprises pacing energy delivery circuitry.

5. The device of claim 1, wherein the control circuitry and detection circuitry are housed in a first can of the plurality of cans, and the energy delivery circuitry is housed in a second can of the plurality of cans.

6. The device of claim 1, wherein a lead is coupled to a first can and a second can of the plurality of cans, the lead comprising a conductor defining a common potential for the first and second cans.

7. The device of claim 1, wherein a lead is coupled to a first can and a second can of the plurality of cans, the lead comprising a control line that couples together the device circuitry respectively housed in the first and second cans.

8. The device of claim 1, wherein a lead is coupled to a first can and a second can of the plurality of cans, the lead comprising a power supply line.

9. The device of claim 1, further comprising a power source provided in one of the plurality of cans.

10. The device of claim 1, further comprising at least two power sources provided in at least two of the plurality of cans.

11. The device of claim 1, further comprising a first power source coupled to the energy delivery circuitry and a second power source coupled to at least the detection and control circuitry.

12. The device of claim 1, wherein each of a first can and a second can of the plurality of cans comprises one or more sense electrodes.

13. The device of claim 1, wherein each of a first can and a second can of the plurality of cans comprises one or more defibrillation electrodes.

14. The device of claim 1, wherein a first can of the plurality of cans is coupled to a second can of the plurality of cans using only a wireless communication coupling.

15. The device of claim 1, wherein a first can of the plurality of cans is coupled to a second can of the plurality of cans using a wireless communication coupling and a lead coupling.

16. The device of claim 1, wherein the cardiac therapy comprises a bi-phasic pulse, a first phase of the bi-phasic pulse delivered by a first can of the plurality of cans and a second phase of the bi-phasic pulse delivered by a second can of the plurality of cans.

17. The device of claim 1, wherein the cardiac therapy comprises a multi-phasic pulse, each phase of the multi-phasic pulse delivered by different ones of the plurality of cans.

18. The device of claim 1, wherein the energy delivery circuitry is provided in each of the plurality of cans, and wherein the control circuitry coordinates delayed energy delivery from one of the plurality of cans relative to other ones of the plurality of cans.

19. The device of claim 1, wherein the energy delivery circuitry is provided in each of the plurality of cans, and wherein the control circuitry coordinates substantially simultaneous energy delivery the plurality of cans.

20. An implantable cardiac device, comprising;
a first can configured for subcutaneous, non-intrathoracic placement relative to a heart of a patient;
a second can configured for subcutaneous, non-intrathoracic placement relative to the patient's heart;
device circuitry housed within and distributed between the first and second cans, the device circuitry comprising at least detection circuitry and control circuitry;
a first energy storage device and first energy delivery circuitry housed in the first can, the first energy delivery circuitry coupled to the first energy storage device and configured to deliver a cardiac therapy to the patient's heart; and
second energy delivery circuitry housed within the second can and configured to deliver a cardiac therapy to the patient in coordination with the first energy delivery circuitry.

21. The device of claim 20, wherein the first and second energy delivery circuitry are conductively coupled in series.

22. The device of claim 20, wherein an output of the first energy delivery circuitry is electrically coupled to at least a portion of the first can, an output of the second energy delivery circuitry is electrically coupled to at least a portion of the second can, and the first and second energy delivery circuitry are conductively coupled in series.

23. The device of claim 20, wherein the first and second energy delivery circuitry are conductively coupled in series, and a combined output defibrillation voltage of the first and second energy delivery circuitry is greater than about 800 Volts.

24. The device of claim 20, wherein an output stage of the first and second energy delivery circuitry comprises a current-limiting field-effect transistor.

25. The device of claim 20, wherein an output stage of the first and second energy delivery circuitry comprises an insulated gate bipolar transistor.

26. The device of claim 20, wherein the first energy delivery circuitry comprises a first insulated gate bipolar transistor, the second energy delivery circuitry comprises a second insulated gate bipolar transistor, and the first and second insulated gate bipolar transistors are connected in series.

27. The device of claim 20, wherein the first energy delivery circuitry comprises a first transistor, the second energy delivery circuitry comprises a second transistor, and the first and second transistors are configured to provide a combined output energy greater than about 40 Joules.

28. The device of claim 20, wherein the first energy delivery circuitry comprises a first transistor, the second energy delivery circuitry comprises a second transistor, and the first and second transistors are configured to provide a combined output energy greater than about 100 Joules.

29. The device of claim 20, wherein the first can is electrically coupled to the second can using a conductor, a potential of the conductor defining a common potential between the first can and the second can.

30. The device of claim 20, wherein the first can is coupled to the second can using only a wireless communication coupling.

31. The device of claim 20, wherein:
the first energy delivery circuitry further comprises:
a first output terminal, a first input terminal, and a first capacitor, the first input terminal coupled to the first capacitor; and
a first switch coupled between the first input terminal and the first output terminal; and
the second energy delivery circuitry further comprises:
a second output terminal, a second input terminal, and a second capacitor, the second input terminal coupled to the second capacitor; and
a second switch coupled between the second input terminal and the second output terminal;
wherein the first output terminal is electrically coupled to the second input terminal.

32. The device of claim 20, wherein:
the first energy delivery circuitry further comprises:
a first input terminal, a first output terminal, and a first capacitor; and
a first switch and a second switch respectively coupled between the first input terminal and the first output terminal; and
the second energy delivery circuitry further comprises:
a second input terminal, a second output terminal, and a second capacitor; and
a third switch and a fourth switch respectively coupled between the second input terminal and the second output terminal;
wherein the first output terminal is electrically coupled to the second input terminal.

33. A method, comprising:
providing a first can of a cardiac therapy delivery device configured for subcutaneous, non-intrathoracic placement relative to a heart of a patient;
providing a second can of the cardiac therapy delivery device configured for subcutaneous, non-intrathoracic placement relative to the patient's heart, each of the first and second cans housing circuitry of the cardiac therapy delivery device; and
delivering a cardiac therapy to the patient's heart using each of the first and second cans.

34. The method of claim 33, wherein the cardiac therapy is delivered by each of the first and second cans concurrently.

35. The method of claim 33, wherein delivering the cardiac therapy comprises delaying energy delivery by the second can relative to energy delivery by the first can.

36. The method of claim 33, wherein one of the first and second cans defines a first electrode for the cardiac therapy delivery device, and the other of the first and second cans defines a second electrode for the cardiac therapy delivery device.

37. The method of claim 33, wherein each of the first and second cans defines an electrode of a first polarity for the cardiac therapy delivery device, and a conductor coupled between the first and second cans defines an electrode of a second polarity for the cardiac therapy delivery device.

38. The method of claim 33, further comprising detecting cardiac activity using one of the first and second cans.

39. The method of claim 33, further comprising detecting cardiac activity using each of the first and second cans.

40. The method of claim 33, further comprising detecting cardiac activity using one of the first and second cans, and delivering the cardiac therapy from the other of the first and second cans.

41. The method of claim 33, further comprising supplying power to the circuitry of the cardiac therapy delivery device from one of the first and second cans.

42. The method of claim 33, further comprising supplying power to the circuitry of the cardiac therapy delivery device from each of the first and second cans.

43. The method of claim 33, further comprising supplying energy for the cardiac therapy delivery from one of the first and second cans.

44. The method of claim 33, further comprising supplying energy for the cardiac therapy delivery from each of the first and second cans.

45. The method of claim 33, further comprising positioning the first and second cans in relation to a heart so that a majority of ventricular tissue is included within a volume defined between the first and second cans.

46. The method of claim 33, further comprising:
positioning the first can in relation to a superior aspect of the heart; and
positioning the second can in relation to an inferior aspect of the heart.

47. The method of claim 33, further comprising positioning one of the first and second cans parallel to a ventricular free wall and extending a predetermined distance beyond an apex of the patient's heart.

48. The method of claim 33, further comprising switchably coupling the first can to the second can for delivering the cardiac therapy.

49. The method of claim 33, further comprising switchably coupling the first can to the second can in series for delivering the cardiac therapy.

50. The method of claim 33, further comprising delivering a shock to the patient's heart, the shock having a potential greater than about 800 Volts.

51. The method of claim 33, further comprising delivering a shock to the patient's heart, the shock having a potential greater than about 1200 Volts.

52. The method of claim 33, further comprising delivering a shock to the patient's heart, the shock having a total energy greater than about 40 Joules.

53. The method of claim 33, further comprising delivering a shock to the patient's heart, the shock having a total energy greater than about 100 Joules.

54. The method of claim 33, further comprising delivering a shock to the patient's heart, the shock comprising a multi-phasic pulse, at least a first phase of the multi-phasic pulse delivered by the first can and at least a second phase of the multi-phasic pulse delivered by the second can.

55. An implantable cardiac device, comprising;
a first can configured for subcutaneous, non-intrathoracic placement relative to a heart of a patient;
a second can configured for subcutaneous, non-intrathoracic placement relative to the patient's heart, each of the first and second cans comprising means for housing circuitry of the implantable cardiac device; and
means for delivering cardiac therapy to a patient's heart using each of the first and second cans.

56. The device of claim 55, wherein the delivering means comprises means for storing and discharging defibrillation therapy energy, the storing and discharging means housed in one of the first and second cans.

57. The device of claim 55, wherein the delivering means comprises means for storing and discharging defibrillation therapy energy, the storing and discharging means housed in each of the first and second cans.

58. The device of claim 55, further comprising means for detecting activity of the heart, the detecting means housed in one of the first and second cans.

59. The device of claim 55, further comprising means for detecting activity of the heart, the detecting means housed in each of the first and second cans.

60. The device of claim 55, further comprising means for detecting activity of the heart, and the delivering means comprises means for storing and discharging defibrillation therapy energy, wherein:
the storing and discharging means is housed in one of the first and second cans; and
the detecting means is housed in the other of the first and second cans.

61. The device of claim 55, wherein the delivering means comprises means for delivering a shock to cardiac tissue included within a volume defined between the first and second cans, the shock having a total energy greater than about 40 Joules.

62. The device of claim 55, wherein the delivering means comprises means for delivering a shock to cardiac tissue included within a volume defined between the first and second cans, the shock having a potential greater than about 800 Volts.
